(19) United States
(12) Patent Application Publication
(10) Pub. No.: US 2026/0294406 A1
(43) Pub. Date: Oct. 1, 2026

(54) ROOTER WITH SELECTIVE MANUAL AND MOTORIZED OPERATION

(71) Applicant: Distal Access, LLC, Park City, UT (US)

(72) Inventors: Kent Beck, Layton, UT (US); Shawn Patrick Fojtik, Park City, UT (US); Tim Nieman, North Salt Lake, UT (US)

(21) Appl. No.: 19/633,998

(22) Filed: Mar. 30, 2026

Related U.S. Application Data
(60) Provisional application No. 63/780,443, filed on Mar. 30, 2025.

(51) Int. Cl. A61B 10/02; F16C 19/10

(52) U.S. Cl. CPC A61B 10/025; F16C 19/10; F16C 2316/10; F16C 2380/27

(57) ABSTRACT

A rooter may have both manual and powered operation capabilities. The rooter includes a trigger that manually rotates a spindle of the rooter and, thus, manually rotates a tool coupled to the spindle as the trigger is moved between a distal position and a partially proximal position. The trigger actuates motorized rotation of the spindle and a tool coupled to the spindle when pulled to a fully proximal position. The rooter may include a mechanism, such as a thrust bearing, that induces vibration in the tool and the rooter. Methods for using rooters with manual and powered operation capabilities and/or thrust bearings are also disclosed.

### CROSS-REFERENCE TO RELATED APPLICATION

A claim for priority to the Mar. 30, 2025, filing date of U.S. Provisional Patent Application No. 63/780,443, titled HAND-OPERATED AND/OR ELECTRIC DRILL AND METHODS OF USE (“the '443 Provisional Application”), is hereby made pursuant to 35 U.S.C. § 119(e). The entire disclosure of the '443 Provisional Application is hereby incorporated herein.

### TECHNICAL FIELD

This disclosure relates generally to rooters that may be used in medical procedures and, more specifically, to a rooter with both manual and powered operation capabilities, as well as a thrust bearing for use with the rooter. Methods for using rooters with manual and powered operation capabilities and/or thrust bearings are also disclosed.

### SUMMARY

In one aspect, a rooter comprises a manually operable rooter that includes a housing, a spindle carried by and rotatable within the housing, a motor, and a trigger. The trigger manually drives rotation of the spindle when depressed to a first position. Upon depressing the trigger further to a second position, the trigger actuates a switch associated with the motor, enabling the motor to drive rotation of the spindle.

The spindle may be indirectly or directly linked to the motor. In embodiments where the spindle is indirectly linked to the motor, a position of the motor on the housing may be offset from a passage through the spindle. In embodiments where the spindle is directly linked to the motor, the motor may be positioned directly behind, or proximal to, the spindle.

The rooter may optionally comprise a ratcheting mechanism, which may enable the rooter to rotate the spindle and the tool in a first rotational direction even when the spindle and the tool cannot rotate in the opposite, second rotational direction. The ratcheting mechanism may also induce vibration in a coupler associated with the spindle and in a tool that has been coupled to the coupler. Such vibration may occur while the motor drives rotation of the spindle.

Optionally, the rooter may include a thrust bearing. The thrust bearing may be positioned on a distal portion of the spindle or distal to the spindle. The thrust bearing may induce longitudinal back-and-forth movement in a tool (e.g., an elongated medical device (e.g., a sampling device, such as a biopsy needle, a needle, a cannula, a trocar, or a catheter; a wire; a macerator; a drill bit; etc.), a drill bit, etc.) coupled to and rotatably driven by the spindle. For example, the thrust bearing may enable the tool to rotate without longitudinally moving the tool back and forth while the tool and a distal side of the thrust bearing rotate below a threshold resistance in rotation. The thrust bearing may induce the longitudinal back-and-forth movement in the tool when the tool and the distal side of the thrust bearing encounter a resistance in rotation that equals or exceeds the threshold resistance in rotation. As another example, the thrust bearing may enable the tool to rotate without longitudinally moving the tool back and forth while the spindle and a proximal side of the thrust bearing rotate below a threshold rotational rate. The longitudinal back-and-forth movement may be induced as the rotational rate of the spindle and the proximal side of the thrust bearing reach or exceed the threshold rotational rate.

A thrust bearing may comprise a hub, a first thrust plate, a bearing cage carrying a plurality of bearings, and a second thrust plate.

The hub may include a proximal end and a distal end. The proximal end of the hub may couple to a distal end of the spindle of the rooter. The proximal end of the hub may enable the spindle to drive rotation of the hub while also enabling the hub to slide longitudinally back-and-forth over the distal end of the spindle. The distal end of the hub may couple to a proximal end of a tool (e.g., an elongated medical device, a drill bit, etc.) that is to be rotated as the hub rotates. A longitudinal position of the tool relative to the hub may be fixed as the proximal end of the tool is coupled to the distal end of the hub; thus, if the hub moves longitudinally relative to a spindle of a rooter, the tool will move longitudinally with the hub to substantially the same extent as the hub (e.g., accounting for a tool with a compressibility that exceeds a compressibility of the hub, etc.) or to the same extent as the hub.

The first thrust plate may also be referred to as a distal thrust plate. The first thrust plate may have a first outer surface (i.e., a distal side) and a first inner surface (i.e., a proximal side). The first inner surface may include one or more first recesses (e.g., dimples, arcuate slots, etc.); in embodiments where the first inner surface includes a plurality of first recesses, the plurality of first recesses may be arranged annularly about a center of rotation of the first thrust plate. The first thrust plate may reside over the hub. A position of the first thrust plate along the hub may be fixed. For example, the first thrust plate may be fixedly secured to the hub. As another example, the first thrust plate may be formed integrally with the hub.

The bearing cage may include a first side (i.e., a distal side) and a second side (i.e., a proximal side). The bearing cage may carry a plurality of bearings. The bearings may protrude from at least the first side of the bearing cage. Optionally, the bearings may also protrude from the second side of the bearing cage. The bearings may be arranged annularly around a center of rotation of the bearing cage. The bearing cage may reside over the hub, proximally adjacent to the first inner surface of the first thrust plate and with the first side of the bearing cage facing the first inner surface of the first thrust bearing plate. The bearing cage may be able to slide longitudinally over the hub.

The second thrust plate may also be referred to as a proximal thrust plate. The second thrust plate may have a second inner surface (i.e., a distal side) and a second outer surface (i.e., a proximal side). Optionally, the second inner surface may include one or more second recesses (e.g., dimples, arcuate slots, etc.); in embodiments where the second inner surface includes a plurality of second recesses, the plurality of second recesses may be arranged annularly about a center of rotation of the second thrust plate. The second thrust plate may reside over the hub, proximally adjacent to the second side of the bearing cage and with the second inner surface of the second thrust plate facing the second side of the bearing cage. The second thrust plate may be able to slide longitudinally over the hub.

The thrust bearing may optionally include a spring. The spring may force the first thrust plate distally, away from one or both of the bearing cage, the bearing(s) carried by the bearing cage, and the second thrust plate.

A retainer may hold the thrust bearing in place relative to the distal end of the spindle in a manner that enables a proximal load to force the first thrust bearing toward the second thrust bearing. Such a retainer may include a distal limiter that may limit the distance the first thrust bearing and the hub may move distally and, thus, may ensure that the thrust bearing remains assembled with the spindle of the rooter. Such a retainer may also include a proximal limiter that may limit the distance the second thrust plate may move proximally toward a remainder of the rooter as the first thrust plate may continue to move proximally toward the bearing cage and the second thrust plate. The retainer may be part of a housing for the thrust bearing or the rooter.

The first recess(es) and any optional second recess(es) may be positioned to rotationally align with the bearings when the first thrust plate, the bearing cage, and the second thrust plate are assembled with the hub. Each of the first recess(es) and optional second recess(es) may receive a bearing. When the bearing(s) is (are) received within first recess(es) and optional second recess(es), the distance between the first thrust plate and the second thrust plate may be minimized and the first thrust plate, by way of the bearing(s) and bearing cage, may engage the second thrust plate in a manner that causes first thrust plate, a tool rotated by the rooter, and the rooter to vibrate.

When the bearing(s) is (are) not received within the first recess(es) and the optional second recess(es), the first thrust plate and second thrust plate may be spaced a maximum distance apart from each other, which pushes the first thrust plate, the hub, and any tool (e.g., an elongated medical device, a screwdriver, a drill, etc.) coupled to the distal end of the hub distally. As the first thrust plate and second thrust plate move repeatedly between the minimum distance and maximum distance, the first thrust plate, the hub, and any tool coupled to the hub vibrate or move in a hammering action.

In another aspect, a method includes introducing an elongated medical device into a target location within a body of a subject. The method may include coupling an elongated medical device, a screwdriver, a drill, or the like to a spindle of a rooter, introducing a distal tip of the elongated medical device into the body of the subject, and advancing the distal tip through the body of the subject to the sample location. Introducing and/or advancing the distal tip may include manually driving the spindle by depressing a trigger of the medical rooter up to a first position to manually rotate the spindle and the sampling device. Introducing and/or advancing the distal tip may further include driving the spindle by depressing the trigger to a second position beyond the first position to enable a motor of the rooter to drive rotation of the spindle and elongated medical device.

The method may further include moving the sampling device longitudinally back and forth while introducing and/or advancing the distal tip. Such back-and-forth movement may occur automatically as the elongated medical device encounters at least a threshold resistance to rotation. More specifically, the longitudinal back-and-forth movement may be induced by a thrust bearing. For example, the longitudinal back-and-forth movement may be induced as the elongated medical instrument and a distal member of the thrust bearing encounter at least a threshold resistance to rotation. As another example, the longitudinal back-and-forth movement may be induced upon spinning the spindle and a proximal side of the thrust bearing at a rotational rate that equals or exceeds a threshold rotational rate.

Moving the elongated medical device longitudinally back and forth may occur while driving rotation of the spindle by manually operating the rooter or while driving rotation of the spindle with a motor.

In some embodiments, the method may include obtaining a sample from the target location within the body of the subject.

Other aspects of the disclosed subject matter, as well as features and advantages of various aspects of the disclosed subject matter, will become apparent to those of ordinary skill in the art through consideration of the ensuing description, the accompanying drawings, and the appended claims.

### BRIEF DESCRIPTION OF THE DRAWINGS

In the drawings:

FIG. 1 is a side view of an embodiment of a rooter of this disclosure, which includes a motor inline with a spindle of the rooter;

FIG. 2 is an orthogonal assembly view of embodiments of various components of the embodiment of the rooter shown in FIG. 1;

FIG. 3 is a side assembly view of the embodiments of the components of the embodiment of the rooter shown in FIG. 2;

FIG. 4 provides an enlarged side assembly view of embodiments of the spindle and gears of the embodiment of the rooter shown in FIG. 1;

FIG. 5 is a cross-sectional representation showing a relationship of the spindle and gears of FIG. 4 during manual operation of the rooter;

FIG. 6 is a cross-sectional representation showing a relationship of the spindle and gears of FIG. 4 during motorized operation of the rooter;

FIG. 7 schematically illustrates an embodiment of a thrust bearing that may be used with a rooter of this disclosure, which includes a hub, a first thrust plate, a bearing cage, bearings, a second thrust plate, and a retainer;

FIG. 8 is an assembly view of embodiments of the first thrust plate, bearing cage, bearings, and second thrust plate shown in FIG. 7, with the first thrust plate and the second thrust plate oriented orthogonally, in different directions;

FIG. 9 is a cross-sectional representation of a portion of a thrust plate, showing an embodiment of a recess in the thrust plate;

FIG. 10 is a cross-sectional representation of a portion of a thrust plate, showing another embodiment of a recess in the thrust plate;

FIG. 11 is a perspective assembly view of other embodiments of the first thrust plate, bearing cage, bearings, and a second thrust plate;

FIG. 12 is a graph showing sound generated during use of a rooter of this disclosure; and

FIG. 13 is a side view of another embodiment of a rooter of this disclosure, which includes a motor out-of-line with a spindle of the rooter.

### DETAILED DESCRIPTION

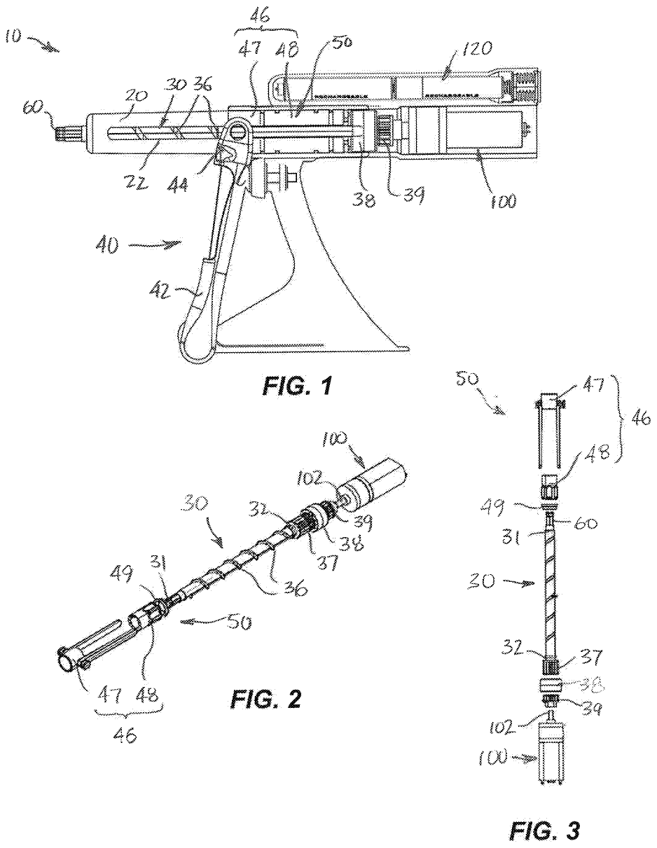

With reference to FIGS. 1-3, an embodiment of a rooter 10 capable of manual and/or motorized operation is depicted. The rooter 10 includes a housing 20, a rotatable element (i.e., a spindle 30) carried by the housing 20, a trigger 40 associated with the housing 20 and the spindle 30 in a manner that enables the trigger 40 to drive rotation of the spindle 30, and a coupler 60 associated with a distal end 31 of the spindle 30. Embodiments of manually driven rooters including these features are disclosed by U.S. Pat. Nos. 8,032,319 and 8,845,621, the entire disclosures of which are hereby incorporated herein. The rooter 10 also includes a motor 100, which may comprise any suitable electric motor (e.g., an electric motor with a shaft 102 that spins at up to 2000 rpm, etc.).

The spindle 30 may be disposed within an interior of the housing 20 in a manner that enables the spindle 30 to spin about its longitudinal axis. As the spindle 30 rotates within the housing 20, which may remain substantially stationary (e.g., within a user's grasp, with vibration of an operating motor, etc.), a tool, such as an elongated medical instrument (e.g., a sampling device, such as a biopsy needle, a needle, a cannula, a trocar, or a catheter; a wire; a macerator; a drill bit; etc.), a screwdriver, or a drill bit engaged by the coupling element, may rotate. In some embodiments, the spindle 30 may comprise an elongated member with a longitudinal axis, about which the spindle 30 may rotate, or spin. In a more specific embodiment, the spindle 30 may include a helical ridge 36, similar to the thread of a bolt or screw, which may enable the spindle 30 to be rotationally driven. The helical ridge 36 may extend from a distal end 31 of the spindle 30 to a location short of a proximal end 32 of the spindle 30.

The trigger 40 may be associated with the spindle 30 in such a way as to cause the spindle 30 to rotate. In a specific embodiment, the trigger 40 may include an external element 42, an intermediate element 44, and an actuator 46. The external element may be configured for manual operation (e.g., engagement and movement by one or more of an individual's fingers, etc.). The intermediate element 44 may extend through an elongated slot 24 in the housing 20. The actuator 46 may move forward and backward, or distally and proximally, along an interior 26 of the housing 20 to interact with the spindle 30.

More specifically, the actuator 46 of the trigger 40 may be disposed around at least a portion of the spindle 30. In embodiments where the spindle 30 has a helical ridge 36, the actuator 46 may be positioned between longitudinally adjacent locations of the helical ridge 36. In other embodiments, the actuator 46 may include one or more grooves that are configured complementarily to the helical ridge 36 and that cooperate with the helical ridge 36. As the external element 42 of the trigger 40 causes the actuator 46 to move over the spindle 30, the actuator 46 and the helical ridge 36 of the spindle 30 may interact with one another to cause the spindle 30, as well as the coupler 60 and any tool (not shown) engaged by the coupler 60, to rotate, or spin. Thus, the trigger 40 may enable manual rotation of the spindle 30, the coupler 60, and a tool that has been coupled to the coupler 60. For example, the trigger 40 may manually rotate the spindle 30 as the external element 42 of the trigger 40 is moved between a distal position, which may also be referred to as a starting position, and a partially proximal position, which may also be referred to herein as a first proximal position.

The trigger 40 may also enable motorized rotation of the spindle 30, the coupler 60, and a tool that has been coupled to the coupler 60. The motor 100 may be located behind, or proximal to, the rotatable element 30. A shaft of the motor 100 may be aligned with the rotatable element 30. As the external element 42 of the trigger 40 pulls the actuator 46 of the trigger 40 to a completely proximal position (i.e., as far proximally as the trigger 40 may be pulled, beyond the proximal extent of the helical ridge 36), which may also be referred to as a second proximal position, the actuator 46 may disengage the helical ridge 36 the spindle 30, couple the motor 100 to the spindle 30, and actuate the motor 100.

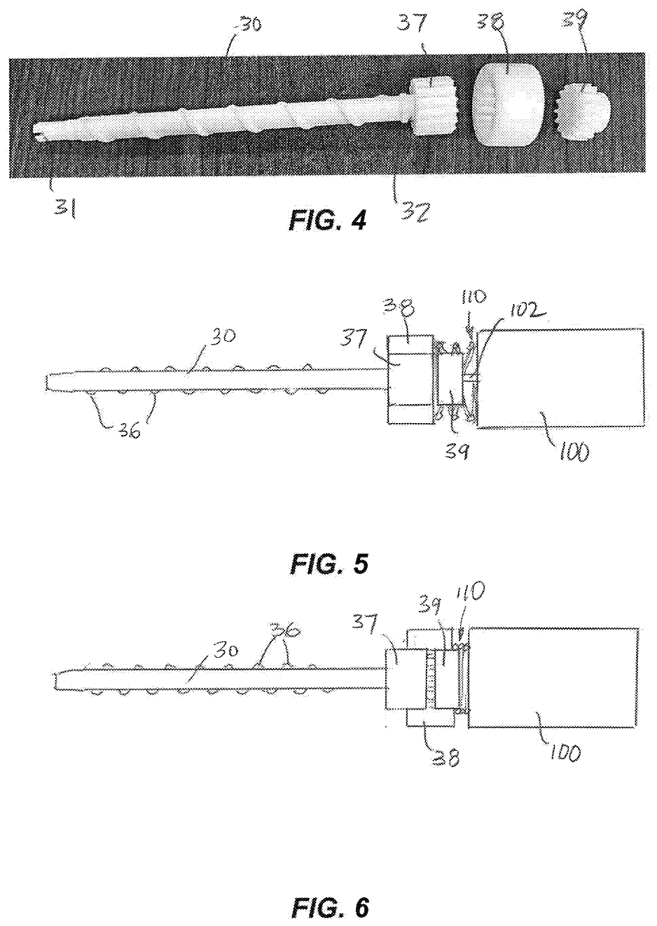

More specifically, with added reference to FIGS. 4-6, the spindle 30 may include a system of gears that enable an individual to switch between manual operation of the spindle 30 and motorized operation of the spindle 30. The spindle 30 may include a spindle gear 37 (e.g., a spline gear, etc.) at its proximal end 32. A motor gear 39 (e.g., a spline gear, etc.) may be provided on the shaft 102 of the motor 100. The spindle gear 37 and the motor gear 39 may have the same diameters, number of teeth, teeth pitch, and teeth shape. The spindle gear 37 and motor gear 39 may be axially aligned with each other. An annular gear 38 that includes interior teeth that are complementary to and mesh with the teeth of the spindle gear 37 and motor gear 39 may slide along but not completely off of the spindle gear 37. The annular gear 38 may also slide onto and off of the motor gear 39. A spring 110 may force the annular gear 38 distally, such that the annular gear 38 resides completely on the spindle gear 37, as shown in FIG. 5. While the trigger 40 (FIGS. 1-3) moves the actuator 46 (FIG. 1-3) between its distal and partially proximal positions (i.e., the first proximal position), the spring 110 keeps the annular gear 38 on the spindle gear 37, as shown in FIG. 5. As the trigger 40 moves the actuator 46 to its completely proximal position (i.e., its second proximal position), however, the proximally directed force on the actuator 46 overcomes the force of the spring 110, enabling the actuator 46, to move proximally beyond and disengage the helical ridge 36 and to move the annular gear 38 proximally such that a proximal portion of the annular gear 38 engages the motor gear 39, as shown in FIG. 6. Additionally, complete proximal movement of the actuator 46 may engage a switch of the motor 100, which may cause the shaft 102 of the motor 100, the motor gear 39, the annular gear 38, and the spindle gear 37 and the spindle 30 to rotate. Once pressure on the trigger 40 is released, the spring may urge the annular gear 38 distally off of the motor gear 39, back to the arrangement shown in FIG. 5, thereby disengaging the spindle 30 from the motor 100.

With returned reference to FIGS. 1-3, the rooter 10 may optionally include a ratcheting mechanism 50 associated with the spindle 30 to enable the spindle 30 to be repeatedly driven in a first rotational direction when resistance on the spindle 30 prevents it from rotating in an opposite, second rotational direction. Embodiments of manually driven rooters with such ratcheting mechanisms are disclosed by U.S. Pat. Nos. 10,352,411, 11,002,346, and 12,390,203, the entire disclosures of which are hereby incorporated herein. More specifically, the ratcheting mechanism 50 may include proximal and distal members with mating teeth that may enable rotation of the spindle 30 in both directions (i.e., forward and reverse; clockwise and counterclockwise) about its longitudinal axis when less than a threshold rotational resistance is present on a rotated tool (not shown) (e.g., an elongated medical instrument, a screwdriver, a drill, etc.) that has been coupled to a coupler 60 of the rooter 10. The ratcheting mechanism 50 may also enable the trigger 40 and its actuator 46 to return to their starting positions, from which the trigger 40 can drive further rotation of the spindle 30 in the first rotational direction, even when the rotational resistance on the rotated tool equals or exceeds the threshold rotational resistance and, thus, prevents the rotated tool from oscillating, or from rotating in a direction (e.g., reverse, counterclockwise, etc.) that would otherwise enable the trigger 40 to return to its starting position.

In some embodiments, the ratcheting mechanism 50 includes a distal member 47 of the actuator 46, a proximal member 48 of the actuator 46, and a biasing member 49 (e.g., a spring, etc.). The distal member 47 may be coupled to the intermediate element 44 of the trigger 40 and may be capable of sliding along a length of the spindle 30 without directly causing the spindle 30 to rotate and capable of engaging the proximal member 48 when the trigger 40 forces the distal member 47 proximally along the length of the spindle 30. The proximal member 48 may be capable of engaging the spindle 30 in a manner that drives rotation of the spindle 30 (e.g., by receiving or engaging a helical ridge 36 of the spindle 30, etc.) as the distal member 47 forces the proximal member 48 proximally along the length of the spindle 30. Upon releasing the trigger 40, the biasing member 49 may force the proximal member 48 and the distal member 47 in a distal direction along the length of the spindle 30. If the force of the biasing element 49 exceeds a rotational resistance on a rotated tool that has been coupled to the coupler 60 (and, thus, to the spindle 30), distal movement of the proximal member 48 of the ratcheting mechanism 50 may drive the spindle 30, the coupler 60, and the rotated tool in a reverse direction (i.e., the second rotational direction), thus enabling oscillation of the coupler 60 and the rotated tool. If the rotational resistance on the rotated tool exceeds the biasing force of the biasing member 49, the proximal member 48 may disengage the distal member 47 of the ratcheting mechanism 50, enabling the biasing member 49 to force the proximal member 48 distally without rotating the spindle 30 (thus, the proximal member 48, rather than the spindle 30, spins when the proximal member 48 moves distally), and forcing the distal member 47 of the ratcheting mechanism 50 and the trigger 40 distally as well.

In embodiments where the rooter 10 includes the optional ratcheting mechanism 50, the motor 100 may spin the spindle 30 with enough power, speed, and/or torque to intermittently overcome the biasing force of the biasing member 49. The proximal member 48 may then rotate relative to the distal member 47, causing the teeth of the proximal member 48 and distal member 47 to repeatedly engage and disengage each other, causing the distal member 47 to bump relative to the proximal member 48, or repeatedly moving the distal member 47 away from and toward the proximal member 48, to provide an axial thrust that may cause the coupler 60 and any rotating tool attached thereto to vibrate.

Such vibration may be felt by the user's hand during actuation. Drilling through a dense substrate will produce more vibration than drilling through less-dense substrate. By way of example and not limitation, a user pushing down on the apparatus overpowering the gear teeth of the distal member 47 of the ratcheting mechanism by driving rotation of the proximal member 48 with the motor 100 while drilling dense bone will provide more tactile and/or haptic feedback than drilling through less dense bone. This difference in tactile and/or haptic feedback can help a user determine a type of bone (or other tissue) the rotating tool is being advanced through (e.g., dense cortical bone, less dense cancellous bone, less dense intramedullary bone, etc.).

With continued reference to FIGS. 1-3, a rooter 10 may include an optional thrust bearing 200. The thrust bearing 200 may comprise an alternative to the optional ratcheting mechanism 50 or the thrust bearing 200 may be used in addition to the optional ratcheting mechanism 50. The thrust bearing 200 may be positioned on a distal portion of the spindle 30 or distal to the spindle 30. The thrust bearing 200 may cause the rooter 10 to vibrate.

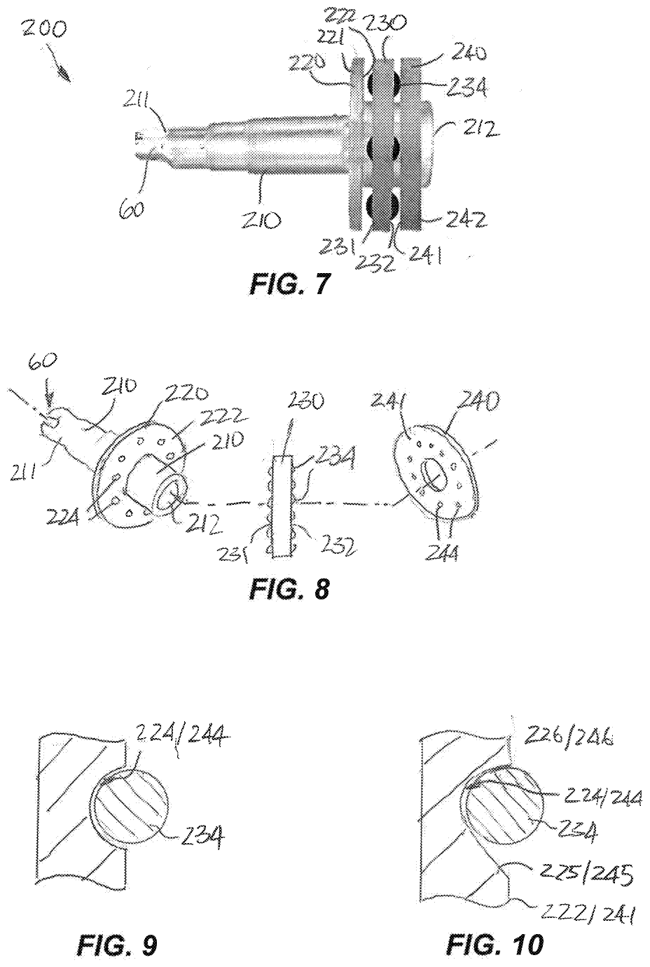

Various embodiments of the thrust bearing 200 are shown in FIGS. 7-11. As shown in FIGS. 7 and 8, the thrust bearing 200 may include a hub 210, a first thrust plate 220, a bearing cage 230, one or more bearings 235 carried by the bearing cage 230, and a second thrust plate 240. The components of the thrust bearing 200 may be held together and in place relative to the spindle 30 (FIGS. 1-3) of the rooter 10 (FIGS. 1-3) by way of a retainer 260.

The hub 210 may include a distal end 211 and a proximal end 212. The proximal end 212 of the hub 210 may couple to a distal end 31 (FIGS. 1-3) of the spindle 30 (FIGS. 1-3) of the rooter 10 (FIGS. 1-3). The proximal end 212 of the hub 210 may enable the spindle 30 to drive rotation of the hub 210 while also enabling the hub 210 to slide longitudinally back-and-forth over the distal end 31 of the spindle 30. The distal end 211 of the hub 210 may couple to a proximal end of a tool (not shown) (e.g., an elongated medical device, a drill bit, etc.) that is to be rotated as the hub 210 rotates. A longitudinal position of the tool relative to the hub 210 may be fixed as the proximal end of the tool is coupled to the coupler 60, which may be defined by or coupled to the distal end 211 of the hub 210; thus, if the hub 210 moves longitudinally relative to a spindle 30 of a rooter 10, the tool will move longitudinally with the hub 210 to substantially the same extent as the hub 210 (e.g., accounting for a tool with a compressibility that exceeds a compressibility of the hub, etc.) or to the same extent as the hub 210.

The first thrust plate 220 may also be referred to as a distal thrust plate. The first thrust plate 220 may have a first outer surface 221 (i.e., a distal side) and a first inner surface 222 (i.e., a proximal side). The first inner surface 222 may include one or more first recesses 224 (e.g., dimples, arcuate slots, etc.); in embodiments where the first inner surface 222 includes a plurality of first recesses 224, the plurality of first recesses 224 may be arranged annularly about a center of rotation of the first thrust plate 220. The first thrust plate 220 may reside over the hub 210. A position of the first thrust plate 220 along the hub 210 may be fixed. For example, the first thrust plate 220 may be fixedly secured to the hub 210. As another example, the first thrust plate 220 may be formed integrally with the hub 210.

The bearing cage 230 may include a first side 231 (i.e., a distal side) and a second side 232 (i.e., a proximal side). The bearing cage 230 may carry a plurality of bearings 234. The bearings 234 may protrude from at least the first side 231 of the bearing cage 230. Optionally, the bearings 234 may also protrude from the second side 232 of the bearing cage. The bearings 234 may be arranged annularly around a center of rotation of the bearing cage 230. The bearing cage 230 may reside over the hub 210, proximally adjacent to the first inner surface 222 of the first thrust plate 220 and with the first side 231 of the bearing cage 230 facing the first inner surface 222 of the first thrust bearing plate 220. The bearing cage 230 may be able to slide longitudinally over the hub 210.

The second thrust plate 240 may also be referred to as a proximal thrust plate. The second thrust plate 240 may have a second inner surface 241 (i.e., a distal side) and a second outer surface 242 (i.e., a proximal side). Optionally, the second inner surface 241 may include one or more second recesses 244 (e.g., dimples, arcuate slots, etc.); in embodiments where the second inner surface 242 includes a plurality of second recesses 244, the plurality of second recesses 244 may be arranged annularly about a center of rotation of the second thrust plate 240. The second thrust plate 240 may reside over the hub 210, proximally adjacent to the second side 232 of the bearing cage 230 and with the second inner surface of the second thrust plate 240 facing the second side 232 of the bearing cage 230. The second thrust plate 240 may be able to slide longitudinally over the hub 210. The second thrust plate 240 may rotate independently of the first thrust plate 220; in some embodiments, the second thrust plate 240 may remain rotationally stationary as the hub 210 and first thrust plate 220 rotate.

The thrust bearing 200 may optionally include a spring 250. The spring 250 may force the first thrust plate 220 distally, away from one or more of the bearing cage 230, the bearing(s) 234 carried by the bearing cage 230, and the second thrust plate 240.

The retainer 260 may hold the thrust bearing 200 in place relative to the distal end 31 (FIGS. 1-3) of the spindle 30 (FIGS. 1-3) of the rooter 10 in a manner that enables a proximal load to force (e.g., the weight of the rooter 10, a drilling force applied by a user to the rooter 10, etc.) the first thrust bearing 220 toward the second thrust bearing 240. Such a retainer 260 may include a distal limiter 262 that may limit the distance the first thrust bearing 220 and the hub 210 may move distally and, thus, may ensure that the thrust bearing 200 remains assembled with the spindle 30 of the rooter 10. Such a retainer 260 may also include a proximal limiter 264 that may limit the distance the second thrust plate 240 may move proximally toward a remainder of the rooter 10 as the first thrust plate 220 may continue to move proximally toward the bearing cage 230 and the second thrust plate 240. The retainer 260 may be part of a housing for the thrust bearing 200 or the housing 20 (FIGS. 1-3) of the rooter 10.

The first recess(es) 224 and any optional second recess(es) 244 may be positioned to rotationally align with the bearings 234 when the first thrust plate 220, the bearing cage 230, and the second thrust plate 240 are assembled with the hub 210. Each of the first recess(es) 224 and optional second recess(es) 244 may receive a bearing 234. When the bearing(s) 234 is (are) received within first recess(es) 224 and optional second recess(es) 244, the distance between the first thrust plate 220 and the second thrust plate 240 may be minimized and the first thrust plate 220, by way of the bearing(s) 234 and bearing cage 230, may engage the second thrust plate 240 in a manner that causes the first thrust plate 220, a tool rotated by the rooter 10 (FIGS. 1-3), and the rooter 10 to vibrate.

For example, when the bearing(s) 234 is (are) not received within the first recess(es) 224 and the optional second recess(es) 244, the first thrust plate 220 and second thrust plate 240 may be spaced a maximum distance apart from each other, which pushes the first thrust plate 220, the hub 210, and any tool (e.g., an elongated medical device, a screwdriver, a drill, etc.) coupled to the distal end 211 of the hub 210 distally. As the first thrust plate 220 and second thrust plate 240 move repeatedly between the minimum distance and maximum distance, the first thrust plate 220, the hub 210, and any tool coupled to the hub vibrate or move in a hammering action.

FIG. 9 shows an embodiment of a recess 224, 244, in which the recess 224, 244 comprises a dimple having the shape of part of a sphere (e.g., a hemispherical shape, a spherical dome shape, etc.). FIG. 10 shows an embodiment of a recess 224, 244, in which the recess has an entry side 225, 245 with a gradual slope and an exit side 226, 246 with a steeper slope or that is oriented substantially perpendicular to the surface 222, 241 in which the recess 224, 244 is formed. The entry side 225, 245 may enable a bearing 234 to smoothly enter the recess 224, 244, while the exit side 226, 246 may cause the bearing 234 to jump or bump as the bearing 234 exits the recess 224, 244.

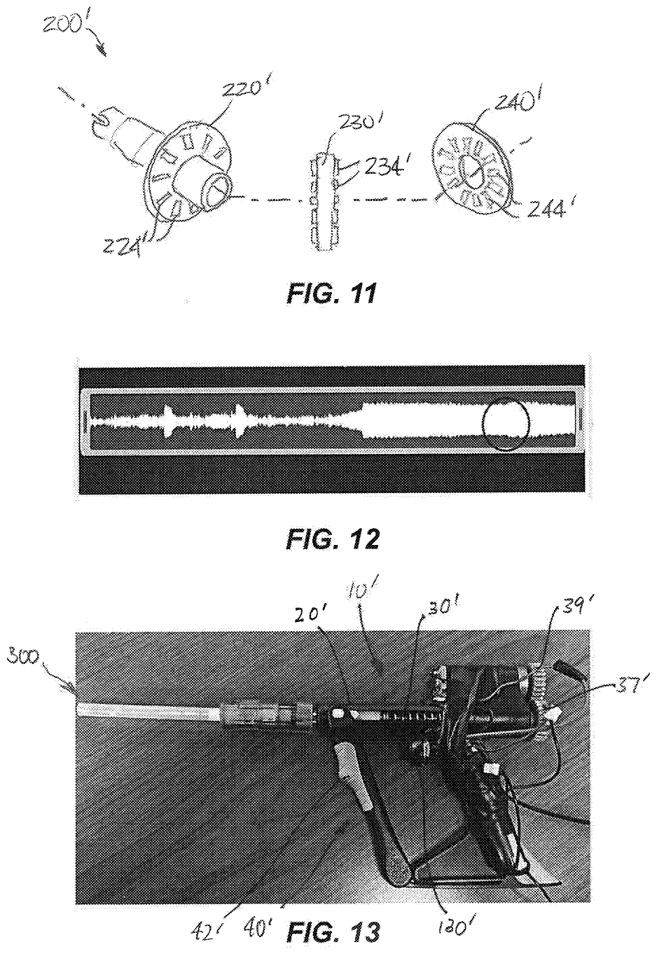

In FIGS. 7-10, the bearings 234 are ball bearings, which are spherical in shape. FIG. 11 is a perspective assembly view of another embodiment of a thrust bearing 200′, which includes a bearing cage 230′ that carries one or more cylindrical bearings 234′, which are also referred to as roller bearings. The first thrust plate 220′ and second thrust plate 240′ include recesses 224′ and 244′, respectively, that are shaped to receive the cylindrical bearings 234′.

With returned reference to FIG. 1, as another option, the rooter 10 may include a power supply 120 be associated with the motor 100. The power supply 120 may be carried by the housing 20 of the rooter 10. Without limitation, the power supply 120 may comprise one or more batteries. Alternatively, power may be supplied to the motor 100 from a remote power source (e.g., an external battery, mains power through an adaptor, etc.).

Referring again to FIGS. 1-3, in use, the rooter 10 enables a user to select between manual operation and motorized operation thereof. The rooter 10 may rotate a tool to facilitate introduction of the tool into and, optionally, through a substrate. When the use is a medical use, the rooter 10 may be used to introduce the tool into the body of a subject.

As the rooter 10 operates, it causes the tool to rotate or spin in a drive direction. The rooter 10 may also cause the rotated tool to spin in a reverse direction (which may enable oscillation of the rotated device). Optionally, depending upon an amount of rotational resistance on the rotated tool (e.g., if at least a threshold amount of rotational resistance to spinning in the reverse direction is present on the rotated tool, etc.), the optional ratcheting mechanism 50 may enable the spindle 30 and the rotated tool to remain stationary, while enabling the distal member 47 and proximal member 48 of the ratcheting mechanism to disengage the helical ridge 36 of the spindle 30 and return to their starting positions (e.g., their distal positions along the spindle 30, etc.).

In some embodiments, movement of the spindle 30 of the rooter 10 and any tool coupled to the coupler 60 associated with the spindle 30 may be accompanied by longitudinal movement of the tool in one or more directions. For example, pressing the rooter 10 and the tool distally into a solid object may result in the application of a proximally directed reactive force to the tool and, thus, to the coupler 60 and spindle 30 of the rooter 10. The proximally directed reactive force may induce longitudinal movement (e.g., vibration, hammering action, etc.) in the tool and in the rooter 10. More specifically, longitudinal movement may be induced with a ratcheting mechanism 50 or a thrust bearing 200 of the rooter 10. Such longitudinal movement (e.g., vibration, hammering action, etc.) may be used alone or in conjunction with oscillation of a rotatable tool to facilitate its introduction into or through a hard structure (e.g., a blockage, such as arterial plaque; a calcification; bone; etc.).

As the graph of FIG. 12 illustrates, the sound of the vibration of the rooter 10 can be distinguished from the sound of the motor 100 of the rooter 10 as a tool accesses a substrate, drills into a substrate, and/or crosses from a substrate of one density to another substrate. Thus, the motorized rooter may provide audible feedback. Specifically, FIG. 12 shows the sound generated while using a rooter 10 of this disclosure. More sound is generated while use of the motor 100 is selected (on the right side of FIG. 12) than when the rooter 10 is manually operated (on the left side of FIG. 12) and as the rooter 10 encounters harder substances. The circled portion of FIG. 12 represents a decrease in sound generated by the rooter 10 as resistance to rotation of the tool decreases, indicating movement of a rotated tool through one layer or structure and into another.

Turning now to FIG. 13, another embodiment of a rooter 10′ is depicted. The rooter 10′ includes a housing 20′, a spindle 30′, a trigger 40′, and a motor 100′. Instead of being aligned with the spindle 30′, a shaft (not shown) of the motor 100′ is carried atop the housing 20′. The trigger 40′ may manually rotate the spindle 30′ as the external element 42′ of the trigger 40′ is moved between a distal position and a partially proximal position. When the trigger 40′ is moved to a completely proximal position, it may depress a switch 130′ (e.g., the illustrated button, etc.), which may actuate the motor 100′. As the motor 100′ is actuated, a motor gear 39′ on the shaft of the motor 100′ may rotate, which may cause a spindle gear 37′ at the proximal end of the spindle 30′, the spindle 30′, and a tool 300 coupled to the spindle 30′ to rotate.

Although this disclosure provides many specifics, these should not be construed as limiting the scope of any of the claims that follow, but merely as providing illustrations of some embodiments of elements and features of the disclosed subject matter. Other embodiments of the disclosed subject matter, and of their elements and features, may be devised which do not depart from the spirit or scope of any of the claims. Features from different embodiments may be employed in combination. Accordingly, the scope of each claim is limited only by its plain language and the legal equivalents thereto.

What is claimed:

1. A rooter, comprising:
a manually operable rooter, including:
a housing;
a spindle carried by the housing and rotatable within the housing;
a trigger that drives rotational movement of the spindle when depressed to a first position; and
a motor:
carried by the housing of the manually operable rooter;
coupled to the spindle to selectively drive the spindle; and
including a switch actuatable by the trigger when depressed to a second position beyond the first position.

2. The rooter of claim 1, wherein the spindle of the manually operable rooter is directly linked to the motor.

3. The rooter of claim 1, wherein the spindle of the manually operable rooter is indirectly linked to the motor.

4. The rooter of claim 3, wherein a position of the motor on the housing is offset from a passage through the spindle.

5. The rooter of claim 1, further comprising:
a ratcheting mechanism along a length of the spindle.

6. The rooter of claim 1, further comprising:
a thrust bearing.

7. The rooter of claim 6, wherein the thrust bearing is positioned on a distal portion of the spindle.

8. The rooter of claim 6, wherein the thrust bearing is positioned distal to the spindle.

9. The rooter of claim 6, wherein the thrust bearing induces longitudinal back-and-forth movement in a sampling device driven by the spindle.

10. The rooter of claim 9, wherein the thrust bearing induces the longitudinal back-and-forth movement in the sampling device upon reaching a threshold resistance to rotation.

11. A method for obtaining a sample from a target location within a body of a subject, comprising:
coupling a sampling device to a spindle of a rooter;
introducing a distal tip of the sampling device into the body of the subject;
advancing the distal tip through the body of the subject to the sample location, including:
manually driving the spindle of the rooter by depressing a trigger of the rooter up to a first position to rotate the spindle and the sampling device to at least partially advance the sampling device toward the target location;
power driving the spindle of the rooter by depressing the trigger of the rooter to a second position beyond the first position to further advance the sampling device toward the target location; and/or
moving the sampling device longitudinally back and forth to further advance the sampling device toward the target location; and
obtaining the sample at the target location with the sampling device.

12. The method of claim 11, wherein advancing the distal tip includes:
manually driving the spindle of the rooter and at least one of power driving the spindle of the rooter and moving the sampling device longitudinally back and forth.

13. The method of claim 11, wherein moving the sampling device longitudinally back and forth occurs while manually operating the rooter.

14. The method of claim 11, wherein moving the sampling device longitudinally back and forth occurs while power driving the spindle of the rooter.

15. The method of claim 11, wherein moving the sampling device longitudinally back and forth comprises inducing longitudinal back and forth movement of the sampling device with a thrust bearing.

16. The method of claim 15, wherein inducing longitudinal back and forth movement of the sampling device with the thrust bearing occurs as the thrust bearing encounters at least a threshold rotational resistance.

17. The method of claim 16, wherein inducing longitudinal back and forth movement of the sampling device with the thrust bearing occurs upon spinning the thrust bearing at a threshold rotational rate.

18. A thrust bearing, comprising:
a first thrust plate comprising a first outer surface and a first inner surface;
a bearing cage carrying a plurality of ball bearings and including a first side and a second side, the ball bearings protruding from the first side and the second side, the first side facing the first inner surface of the first thrust plate; and
a second thrust plate comprising a second inner surface and a second outer surface, the second inner surface facing the second side of the bearing cage,
at least one of the first inner surface of the first thrust plate and the second inner surface of the second thrust plate including at least one recess that can receive a portion of a ball bearing of the plurality of ball bearings.

19. The thrust bearing of claim 18, wherein each of the first inner surface of the first thrust plate and the second inner surface of the second thrust plate includes at least one recess.

20. The thrust bearing of claim 18, wherein the first inner surface of the first thrust plate and/or the second inner surface of the second thrust plate includes a plurality of recesses, a number of the plurality of recesses being the same as a number of the plurality of ball bearings.

21. The thrust bearing of claim 20, wherein each of the first inner surface of the first thrust plate and the second inner surface of the second thrust plate includes a plurality of recesses.

22. The thrust bearing of claim 18, wherein the first thrust plate rotates independently from the second thrust plate.
